(19) United States
(12) Patent Application Publication
(10) Pub. No.: US 2021/0316123 A1
(43) Pub. Date: Oct. 14, 2021

(54) Facemask as a dosage form to administer active ingredient for therapy

(71) Applicants: Isaac Guan, Hong Kong (HK); Yuk Fan Tsao, Hong Kong (HK)

(72) Inventors: Isaac Guan, Hong Kong (HK); Yuk Fan Tsao, Hong Kong (HK)

(21) Appl. No.: 16/846,282

(22) Filed: Apr. 11, 2020

(51) Int. Cl. A61M 35/00; A41D 13/11

(52) U.S. Cl. CPC A61M 35/10; A41D 13/1161

(57) ABSTRACT

Active ingredients that may be used in pharmaceutical products, natural medicines or cosmetic products may be added to the facemask for therapy. In this way the facemask can act as a novel dosage form.

As the active ingredient in the facemask is breathed into the wearer or comes into contact with the wearer's skin, the active ingredient may be absorbed by the wearer in the skin, the respiratory system, or even into the blood circulation system. Using the facemask as a dosage form and route of administration allows for substantial release of active ingredient over a period of time, which maintains a longer therapeutic effect and reduces the risk of an overdose of the active ingredient, which may occur in an one-off or interval mode of absorption.

Furthermore, the facemask is a convenient dosage form and has a pleasing outward appearance. Also, this novel dosage form can encourage patient compliance.

### TECHNICAL FIELD

A facemask, as a dosage form for the administration of active ingredient (which may be used in pharmaceutical products, natural medicines or cosmetic products) into the human body.

### BACKGROUND

There are different routes of administration that active ingredients in pharmaceutical products, natural medicines or cosmetic products can enter into the human body, such as topical, enteral and parenteral. Each type of route of administration has their corresponding dosage forms. Most of these conventional routes of administration cannot maintain a substantial release of the active ingredient over a period of time, such as oral intake or oral or nasal spraying. While the topical dosage form and the intravenous drip can maintain such substantial release, they have their own limitations. Topical dosage form can only be applied on the skin and may not be outwardly pleasing when applied on the face, while intravenous drip is invasive, inconvenient and needs to be administered by medical professionals. Thus, other routes of administration and novel dosage forms which also enables substantial release of active ingredients and overcome the above limitations is desired.

A facemask is typically used to protect the wearer from external undesirable elements, such as dust, smoke, pollen, bacteria, viruses, harmful chemicals and odor. Alternatively, a facemask can be used as a part of fashion. However, the use of a facemask as a novel dosage form and the advantage of a facemask as a route of administration is unexplored.

### BRIEF SUMMARY

Active ingredient intended for therapeutic effect is added to the facemask so that the said facemask can act as a novel dosage form. Active ingredients that may be used in pharmaceutical products, natural medicines or cosmetic products may be added to the facemask. When the wearer puts on the facemask, it should cover the nose, the mouth and a portion of the face of the wearer. The facemask may be adapted to filtering external undesirable elements (such as dust, smoke, pollen, bacteria, virus, harmful chemical, odor and UV radiation), which may enhance the therapeutic effect.

The active ingredient may be released from the facemask and absorbed by the wearer through the wearer's breathing (in vapour and/or fine particle form), through direct contact with the wearer's facial skin, or by a combination of both. The active ingredient may reach the wearer's skin, respiratory system, or through the respiratory system, reaches the blood circulation system, through which reaches to other parts of the wearer's body.

Using the facemask as a dosage form and route of administration may maintain a substantial release of active ingredient over a period of time by the continuous breathing of the wearer or the continuous contact with the wearer's skin. One of the benefits is that rather than having all or a large portion of the active ingredient released in one go (for example in oral intake or oral or nasal spraying), the therapeutic effect can be maintained for a longer period of time. Another benefit is that it reduces the risk of an overdose of the active ingredient, which may occur in an one-off or interval mode of absorption.

Furthermore, the above therapeutic benefits can be easily and conveniently attained as the wearer can put on this facemask and at the same time conduct his or her daily normal activities. Also, facemask as a dosage form has a better outward appearance than other usual topical treatment applied on the face and it can be incorporated into a person's fashion style.

Facemask as a dosage form can also encourage patient compliance. Firstly, it is a novel dosage form which some patients may find it more comfortable and its administration requires little or no difficulty. Secondly, the patient's compliance is encouraged by the better therapeutic result as achieved by this dosage form in certain therapies. Thirdly, it is a convenient dosage form with a more pleasing outlook.

### BRIEF DESCRIPTION OF THE DRAWING

FIG. 1 is a frontal view of a facemask when put on the wearer's face.

FIG. 2 is a side view of the facemask in FIG. 1 when put on the wearer' face.

FIG. 3 shows the inner side of the facemask in FIG. 1

FIG. 4 shows the facemask in FIG. 1 packed inside an individual package

### DETAILED DESCRIPTION

In this specification and the appended claims, the singular forms “a”, “an”, and “the” include plural reference unless the context clearly dictates otherwise. Unless defined otherwise, all technical and scientific terms used herein have the same meaning as commonly understood or generally accepted by one of ordinary skill in the art to which this invention pertains.

The term “active ingredient” refers to any substance or combination of substances, which when absorbed into a human body, may be used for or is intended for preventing, curing or alleviating a disease, a symptom of a disease, ailment, defect or injury in a human being, or for influencing, inhibiting, modifying or regulating the functional states of the human body. Thus, the term “active ingredient” used herein includes active ingredients that may be used in pharmaceutical products, natural medicines or cosmetic products, which when used alone or in a combination, may be used for or is intended for achieving one or more of the above therapeutic effects.

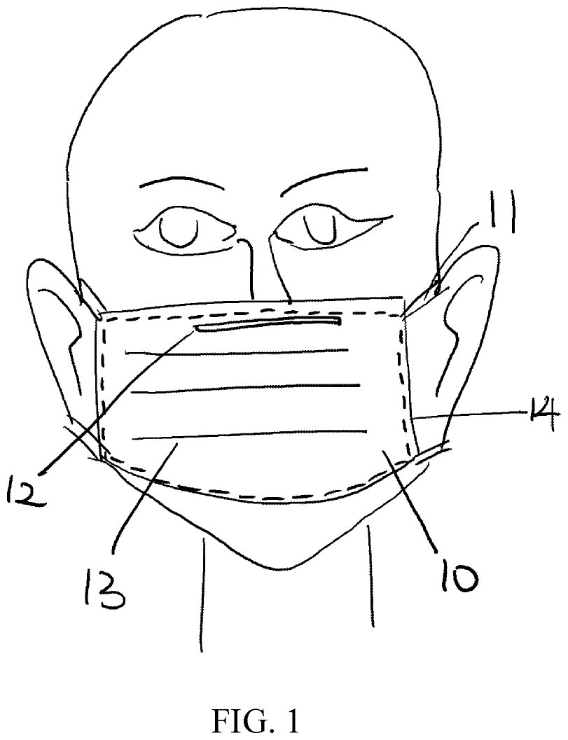

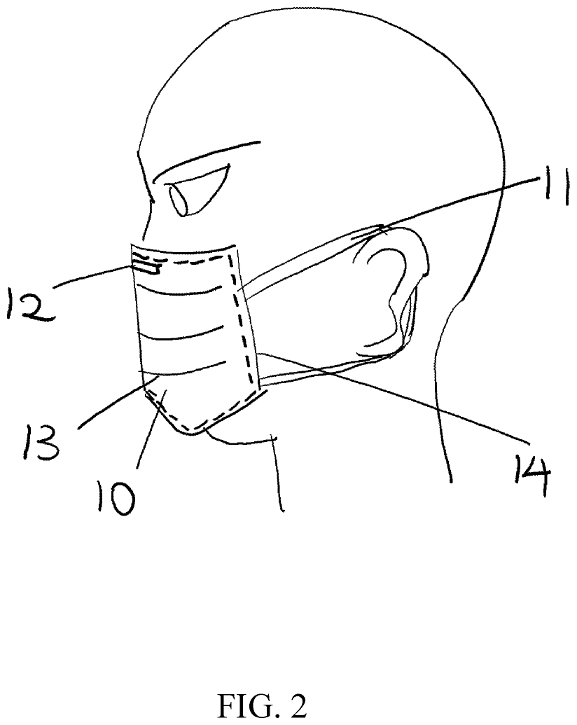

The facemask described herein should cover the nose, the mouth and a portion of the face of the wearer, for example as in FIGS. 1 and 2, which show a certain embodiment of such facemask with a covering portion 10. The covering portion of the facemask 10 is preferably made of fibrous material, artificial fiber (such as glass fiber, cellulose fiber, and polyester fiber) or organic fiber (such as cotton and wool). Preferably, the fibrous material is air permeable to facilitate the wearer's breathing. This is firstly for the wearer's comfort, and secondly, which will be mentioned below, breathing may be one of the means for absorption of the active ingredient into the human body.

Preferably the covering portion 10 of the facemask is able to filter out external undesirable elements (such as dust, smoke, pollen, bacteria, virus, harmful chemical, odor and UV radiation) by physical and/or chemical means. As will be explained below, this may enhance the therapeutic effect. However, facemask without filtering function is also contemplated.

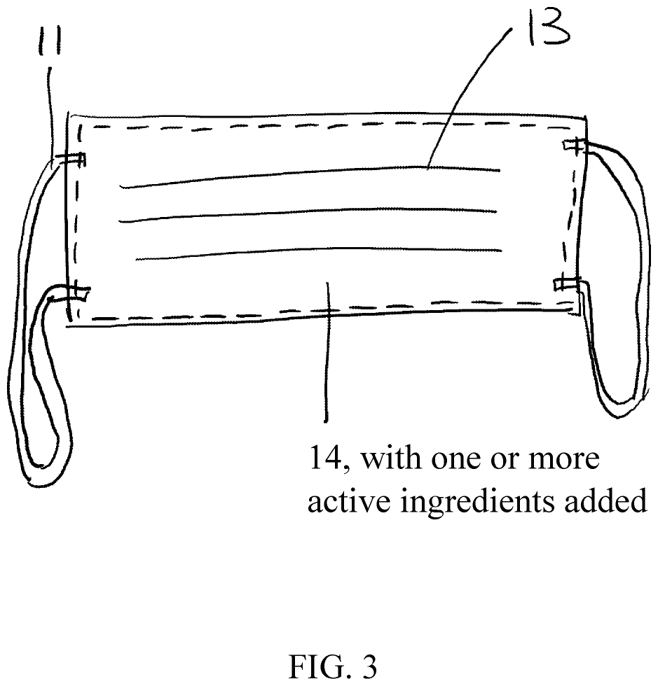

One or more active ingredients (which includes those that may be used in pharmaceutical products, natural medicines or cosmetic products as abovementioned) is added to the facemask, by coating it onto or embedding it into the covering portion 10 of the facemask, or by a combination of both. There are various procedures in the industry to add the active ingredient, such as by spraying the active ingredient onto the facemask or the facemask material. Another example is by immersing the facemask or the facemask material into liquid, semi-liquid or gas that contains the active ingredient. In this way the facemask can act as a novel dosage form. Preferably, the active ingredient should be added to the inner side of the covering portion 14 of the facemask (for example as shown in FIG. 3). This would be easier for the wearer to breathe in the active ingredient or for the active ingredient to come into direct contact with the wearer's skin, depending on the intended means of absorption. However, further or in the alternative, adding the active ingredient to the outer side or the middle layer of the covering portion of the facemask is also contemplated.

The facemask may be mounted onto the wearer's face by ear loops 11, head straps, or other means. The ear loops or head straps may be made of elastic cords (for example as shown in 11), strings intended for tying, or other materials. In certain embodiments, a metal piece or other rigid material 12 may be added to the covering portion 10 near to the wearer's nose to better fit the facemask onto the wearer's nose. In certain embodiments, some of the fibrous material on the covering portion 10 may be folded 13, so that when the facemask is put on, the wearer can unfold and extend it 13 to cover more portion of the wearer's face and to better fit the wearer's face. A facemask that fits the wearer's face well can enhance the absorption of the active ingredient through better contact with wearer skin or by facilitating the breathing in of the active ingredient.

The active ingredient can be released from the facemask and absorbed by the wearer by one of the following means, or by a combination thereof, depending on the intended means of absorption and the physical and chemical property of the active ingredient.

The first means of release and absorption is that as the facemask is mounted on the wearer's face, the wearer is breathing through the facemask; and by the wearer's breathing action, the wearer's body temperature and the active ingredient's dispersibility, the active ingredient is released from the facemask (in vapour form and/or in fine particle form) and breathed in by the wearer. The active ingredient can enter the wearer's body through the wearer's nose or mouth. Upon the initial entry, the active ingredient can be absorbed at, including but not limited to, the nasal cavity, the oral cavity, the throat, the trachea, the bronchus, the lungs, and through the alveolus of the lungs, further into the blood circulation system, which then can reach the various parts of the body.

Thus, given the above-mentioned route of administration, it is preferred to use active ingredient that is targeted for treatment at parts of or the whole of the respiratory system. Also, as mentioned above, the active ingredient can enter the blood circulation system through the lungs, thus the active ingredient intended for treatment at other parts of the human body beyond the respiratory system is also contemplated.

For example, depending on the desired therapeutic effect and the active ingredient used, using the above means of absorption, active ingredient can reach the nasal cavity for treating rhinitis, the oral cavity for treating mouth ulcer, the throat for relieving sore throat, the trachea and the bronchus for relieving asthma, or through the alveolus then enter into the blood circulation for various systematic effects over the human body. There are many commonly known active ingredients for the above examples of desired therapeutic effect, such as mint oil for relieving stuffy nose, steroid for treating rhinitis, ipratropium for treating asthma, hydrogen peroxide for treating mouth ulcer, paracetamol for treating sore throat and digoxin for treating heart failure.

One of the benefits of the above-mentioned route of administration is that the active ingredient can be substantially released from the facemask over a period of time by the continuous breathing of the wearer. Thus, rather than having all or a large portion of the active ingredient released in one go (for example in oral intake or oral or nasal spraying), a substantial release of the active ingredient can be maintained over a period of time. A benefit of such substantial release is that the therapeutic period can be maintained for a longer time. Another benefit is that since the release of the active ingredient is spread over a longer period of time, the risk of an overdose of the active ingredient that may occur in an one-off or interval mode of absorption, is reduced.

Allergic rhinitis (also known hay fever) is triggered by the body's allergic reaction with environmental allergens such as pollen, dust, pet hair, smoke, and mold. Such allergic reaction may cause inflammation; bacteria or virus may invade these vulnerable points in the body and further cause infection. To further illustrate on the example of treating rhinitis mentioned in paragraph 17 above, the therapeutic effect for treating allergic rhinitis is enhanced should the facemask be adapted to filter out undesirable external elements, such as the above environmental allergens, bacteria and virus. It can serve multiple functions in treating a patient who is suffering from allergic rhinitis. Firstly filtering out the environmental allergens to prevent further allergic reaction, secondly filtering out the bacteria and the virus to prevent infection, and thirdly releasing active ingredient to alleviate the inflammation and/or the symptoms (such as stuffy nose, running nose, and itchy nose) as caused by the body's allergic reaction. The aetiology for asthma is similar to that of allergic rhinitis. Thus, should the facemask have the above filtering function, these multiple functions can also be achieved in the example of treating asthma as mentioned in paragraph 17 above or in other ailments that share the similar aetiology.

The second means of release and absorption is when the active ingredient comes into direct contact with the wearer's facial skin. The active ingredient can then be absorbed through the skin for its therapeutic effect.

Some of the application example are listed below. Antibiotics for treating facial acne; cosmetic ingredient for facial therapy, such as moisturizing, hydrating, anti-aging, brightening, depigmentation, scar healing or pores cleaning; also, Danggui extracts applied on the skin can enhance the skin textures and colour according to the Chinese medicine.

Like topical administration, this route of administration also shares the benefit of maintaining a substantial release of the active ingredient over a period of time. If the active ingredient is volatile, the covering portion of the facemask can hold the active ingredient by maintaining the appropriate evaporation rate of the active ingredient.

There are various triggers for causing acne or worsening a pre-existing acne lesion, such as pathogen (e.g. bacteria) and mechanical obstruction of hair follicle from the surrounding environment (e.g. dust). To further illustrate on the example of treating facial acne as mentioned in paragraph 21 above, the therapeutic effect is enhanced should the facemask be adapted to filter out undesirable external elements, such as dust, bacteria and virus. It can serve multiple functions in treating a patient who is suffering from facial acne. Firstly filtering out the bacteria, virus and/or dust to prevent further infection or inflammation at the acne lesion, and secondly releasing active ingredient to kill the bacteria and/or to alleviate symptoms (such as swollenness, itching, and pain) as caused by the inflammation. The multiple functions as achieved by the facemask with filtering function can also be applied to other facial therapies, including but not limited to the examples listed as in paragraph 21 above.

Exposure to UV radiation may cause various undesirable effects on the human skin, such as skin pigmentation, skin tone change, sunburn, skin damage or aging, skin dehydration and trigger or worsen skin inflammation. The UV radiation is one of the undesirable external elements can that be blocked or reduced by the facemask, since its composition materials reduces the light transparency. Thus, this is another helpful function of the facemask to add to its facial therapy, including but not limited to the examples listed in paragraph 21 above.

Furthermore, the wearer can easily and conveniently attain above therapeutic benefits (whether the route of administration is through breathing and/or skin contact). The wearer can simply put on this facemask for therapy and at the same time carry on his or her daily normal activities, whether it be indoor or outdoor. Also, the usual topical administration on the face, such as by applying cream or a patch with active ingredient on the face, is not appealing in outward appearance. Topical administration on the face through a facemask, on the other hand, is pleasing in outward appearance and can be incorporated into a person's fashion style.

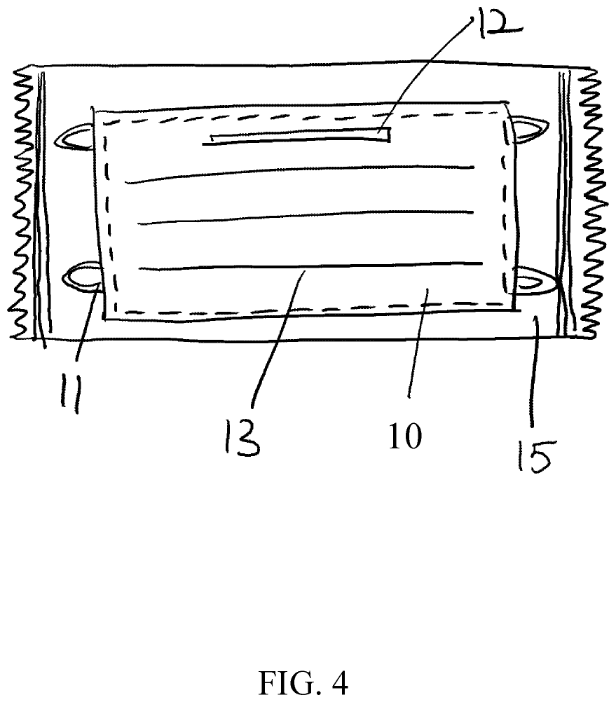

Should the active ingredient be volatile, it is preferred to seal the facemask in an individual package 15 to prevent its exposure in the air before use, for example as shown in a certain embodiment of the package in FIG. 4.

Using the facemask as a dosage form can encourage patient compliance. Firstly, it is a novel dosage form and route of administration, which provides an additional choice or option for patient to take medicine. Some patients are averse to needle injection; some have difficulty in practicing how to use an inhaler and inhaling the spray at the moment it is released; some are unable to or uncomfortable with swallowing pills; and some feel discomfort with certain types of pills in the stomach. Facemask as a dosage form on the other hand may be more comfortable to some patients and its administration requires little or no difficulty. Especially through the COVID-19 pandemic, people are more used to and comfortable with wearing a facemask.

Secondly, the patient's compliance may be encouraged by the better therapeutic result as achieved by the facemask dosage in certain therapies. The active ingredient can be directly absorbed at the skin or the respiratory system. The substantial release of active ingredient can be maintained over a period of time for longer therapeutic effect and reduced risk of overdose. Also, the wearer can benefit from multiple therapeutic functions produced by the facemask dosage form.

Thirdly, some facial topical administration, such as applying a facial mask, often requires the wearer to lie in the bed and cease from other works. Facemask, on the other hand, is comparatively convenient and time saving, as the wearer can put on the facemask and at the same time conduct his or her daily normal activities. Also, some patient may find the conventional facial topical treatment aesthetically unpleasing, especially in front of others. Facemask, on the other hand, has a more pleasing outward appearance, especially through the COVID-19 pandemic, people are more receptive towards others wearing the facemask in the public. All these factors may encourage and contribute to better compliance by the patients or certain group of patients.

What is claimed is:

1. A facemask to which one or more active ingredients is added so that the said facemask acts as a dosage form.

2. The facemask of claim 1 is adapted to cover the nose, the mouth, and a portion of the face of the wearer.

3. The facemask of claim 1, which may be adapted to filter out external undesirable elements.

4. The facemask of claim 1, wherein the said active ingredient is or intended to be absorbed by the wearer for one or more therapeutic effects to the said wearer.

5. The facemask of claim 1, wherein the said active ingredient includes the active ingredients that may be used in pharmaceutical products, natural medicines, or cosmetic products.

6. The facemask of claim 1, wherein the said active ingredient may be released from the said facemask.

7. The facemask of claim 1, wherein the said active ingredient may be absorbed by the wearer of the said facemask when:
(a) the said wearer breathes in the said active ingredient;
(b) the said active ingredient comes into contact with the skin of the said wearer; or
(c) by a combination of the above.

8. The facemask of claim 1, wherein the said active ingredient may reach the following areas:
(a) parts of or the whole of the said wearer's respiratory system;
(b) the facial skin of the said wearer;
(c) any other parts of the said wearer's body through the wearer's respiratory system; or
(d) a combination of any of the above.

9. The facemask of claim 1, wherein the said active ingredient may be substantially released from the said facemask over a period of time.

10. The facemask of claim 1, wherein the said active ingredient is released in a way that reduces the risk of an overdose of the said active ingredient, which may occur in an one-off or interval mode of absorption.
